SEQUENCE LISTING (26 sequences)

SEQ ID NO: 1 - protein, 23 aa, Homo sapiens
IEEQAKTFLDKFNHEAEDLFYQS

SEQ ID NO: 2 - protein, 23 aa, Artificial sequence
  Synthetic peptide
  misc_feature (1)..(1): Xaa can be any amino acid
  misc_feature (5)..(5): Xaa can be any amino acid
XEEQXKTFLDKFNHEAEDLFYQS

SEQ ID NO: 3 - protein, 23 aa, Artificial sequence
  Synthetic peptide
  misc_feature (2)..(2): Xaa can be any amino acid
  misc_feature (6)..(6): Xaa can be any amino acid
IXEQAXTFLDKFNHEAEDLFYQS

SEQ ID NO: 4 - protein, 23 aa, Artificial sequence
  Synthetic peptide
  misc_feature (8)..(8): Xaa can be any amino acid
  misc_feature (12)..(12): Xaa can be any amino acid
IEEQAKTXLDKXNHEAEDLFYQS

SEQ ID NO: 5 - protein, 23 aa, Artificial sequence
  Synthetic peptide
  misc_feature (9)..(9): Xaa can be any amino acid
  misc_feature (13)..(13): Xaa can be any amino acid
IEEQAKTFXDKFXHEAEDLFYQS

SEQ ID NO: 6 - protein, 23 aa, Artificial sequence
  Synthetic peptide
  misc_feature (12)..(12): Xaa can be any amino acid
  misc_feature (16)..(16): Xaa can be any amino acid
IEEQAKTFLDKXNHEXEDLFYQS

SEQ ID NO: 7 - protein, 23 aa, Artificial sequence
  Synthetic peptide
  misc_feature (16)..(16): Xaa can be any amino acid
  misc_feature (20)..(20): Xaa can be any amino acid
IEEQAKTFLDKFNHEXEDLXYQS

SEQ ID NO: 8 - protein, 23 aa, Artificial sequence
  Synthetic peptide
  misc_feature (1)..(1): Xaa can be any amino acid
  misc_feature (5)..(5): Xaa can be any amino acid
  misc_feature (12)..(12): Xaa can be any amino acid
  misc_feature (16)..(16): Xaa can be any amino acid
XEEQXKTFLDKXNHEXEDLFYQS

SEQ ID NO: 9 - protein, 23 aa, Artificial sequence
  Synthetic peptide
  misc_feature (1)..(1): Xaa can be any amino acid
  misc_feature (5)..(5): Xaa can be any amino acid
  misc_feature (16)..(16): Xaa can be any amino acid
  misc_feature (20)..(20): Xaa can be any amino acid
XEEQXKTFLDKFNHEXEDLXYQS

SEQ ID NO: 10 - protein, 23 aa, Artificial sequence
  Synthetic peptide
  misc_feature (9)..(9): Xaa can be any amino acid
  misc_feature (13)..(13): Xaa can be any amino acid
  misc_feature (16)..(16): Xaa can be any amino acid
  misc_feature (20)..(20): Xaa can be any amino acid
IEEQAKTFXDKFXHEXEDLXYQS

SEQ ID NO: 11 - protein, 23 aa, Artificial sequence
  Synthetic peptide
  misc_feature (8)..(8): Xaa can be any amino acid
  misc_feature (12)..(12): Xaa can be any amino acid
  misc_feature (16)..(16): Xaa can be any amino acid
  misc_feature (20)..(20): Xaa can be any amino acid
IEEQAKTXLDKXNHEXEDLXYQS

SEQ ID NO: 12 - protein, 23 aa, Artificial sequence
  Synthetic peptide
  misc_feature (12)..(12): Xaa can be any amino acid
  misc_feature (20)..(20): Xaa can be any amino acid
IEEQAKTFLDKXNHEZEDLXYQS

SEQ ID NO: 13 - protein, 23 aa, Artificial sequence
  Synthetic peptide
  misc_feature (8)..(8): Xaa can be any amino acid
  misc_feature (16)..(16): Xaa can be any amino acid
IEEQAKTXLDKZNHEXEDLFYQS

SEQ ID NO: 14 - protein, 27 aa, Homo sapiens
STIEEQAKTFLDKFNHEAEDLFYQSSL

SEQ ID NO: 15 - protein, 27 aa, Artificial sequence
  Synthetic peptide
  misc_feature (3)..(3): Xaa can be any amino acid
  misc_feature (7)..(7): Xaa can be any amino acid
  misc_feature (14)..(14): Xaa can be any amino acid
  misc_feature (18)..(18): Xaa can be any amino acid
STXEEQXKTFLDKXNHEXEDLFYQSSL

SEQ ID NO: 16 - protein, 27 aa, Artificial sequence
  Synthetic peptide
  misc_feature (3)..(3): Xaa can be any amino acid
  misc_feature (7)..(7): Xaa can be any amino acid
  misc_feature (14)..(14): Xaa can be any amino acid
  misc_feature (18)..(18): Xaa can be any amino acid
STXEEQXKTNLDKXNHEXEDLFYQSSL

SEQ ID NO: 17 - protein, 27 aa, Artificial sequence
  Synthetic peptide
  misc_feature (3)..(3): Xaa can be any amino acid
  misc_feature (7)..(7): Xaa can be any amino acid
  misc_feature (14)..(14): Xaa can be any amino acid
  misc_feature (18)..(18): Xaa can be any amino acid
STXEEQXKLFLDKXNHEXEDLFYQSSL

SEQ ID NO: 18 - protein, 27 aa, Artificial sequence
  Synthetic peptide
  misc_feature (3)..(3): Xaa can be any amino acid
  misc_feature (7)..(7): Xaa can be any amino acid
  misc_feature (14)..(14): Xaa can be any amino acid
  misc_feature (18)..(18): Xaa can be any amino acid
STXEEQXKTFLEKXNHEXEDLFYQSSL

SEQ ID NO: 19 - protein, 27 aa, Artificial sequence
  Synthetic peptide
  misc_feature (3)..(3): Xaa can be any amino acid
  misc_feature (7)..(7): Xaa can be any amino acid
  misc_feature (14)..(14): Xaa can be any amino acid
  misc_feature (18)..(18): Xaa can be any amino acid
STXEEQXKTFLDWXNHEXEDLFYQSSL

SEQ ID NO: 20 - protein, 27 aa, Artificial sequence
  Synthetic peptide
  misc_feature (4)..(4): Xaa can be any amino acid
  misc_feature (8)..(8): Xaa can be any amino acid
STIXEQAXTFLDKFNHEAEDLFYQSSL

SEQ ID NO: 21 - protein, 27 aa, Artificial sequence
  Synthetic peptide
  misc_feature (4)..(4): Xaa can be any amino acid
  misc_feature (8)..(8): Xaa can be any amino acid
  misc_feature (22)..(22): Xaa can be any amino acid
  misc_feature (26)..(26): Xaa can be any amino acid
STIXEQAXTFLDKFNHEAEDLXYQSXL

SEQ ID NO: 22 - protein, 27 aa, Artificial sequence
  Synthetic peptide
  misc_feature (4)..(4): Xaa can be any amino acid
  misc_feature (8)..(8): Xaa can be any amino acid
  misc_feature (22)..(22): Xaa can be any amino acid
  misc_feature (26)..(26): Xaa can be any amino acid
STIXEQAXTNLDKFNHEAEDLXYQSXL

SEQ ID NO: 23 - protein, 27 aa, Artificial sequence
  Synthetic peptide
  misc_feature (4)..(4): Xaa can be any amino acid
  misc_feature (8)..(8): Xaa can be any amino acid
  misc_feature (22)..(22): Xaa can be any amino acid
  misc_feature (26)..(26): Xaa can be any amino acid
STIXEQAXLFLDKFNHEAEDLXYQSXL

SEQ ID NO: 24 - protein, 27 aa, Artificial sequence
  Synthetic peptide
  misc_feature (4)..(4): Xaa can be any amino acid
  misc_feature (8)..(8): Xaa can be any amino acid
  misc_feature (22)..(22): Xaa can be any amino acid
  misc_feature (26)..(26): Xaa can be any amino acid
STIXEQAXTFLEKFNHEAEDLXYQSXL

SEQ ID NO: 25 - protein, 27 aa, Artificial sequence
  Synthetic peptide
  misc_feature (4)..(4): Xaa can be any amino acid
  misc_feature (8)..(8): Xaa can be any amino acid
  misc_feature (22)..(22): Xaa can be any amino acid
  misc_feature (26)..(26): Xaa can be any amino acid
STIXEQAXTFLDWFNHEAEDLXYQSXL

SEQ ID NO: 26 - protein, 35 aa, Homo sapiens
IEEQAKTFLDKFNHEAEDLFYQSSLASWNYNTNIT

(19) United States
(12) Patent Application Publication
(10) Pub. No.: US 2024/0052330 A1
(43) Pub. Date: Feb. 15, 2024

(54) BLOCKADE OF SARS-COV-2 INFECTION USING HYDROCARBON STAPLED PEPTIDES

(71) Applicant: The University of Chicago, Chicago, IL (US)

(72) Inventors: Yu TIAN, Chicago, IL (US); Sicheng JIANG, Chicago, IL (US); Jeffrey A. HUBBELL, Chicago, IL (US); Matthew A. TIRRELL, Chicago, IL (US); James L. LABELLE, Chicago, IL (US)

(21) Appl. No.: 18/257,257

(22) PCT Filed: Dec. 22, 2021
(86) PCT No.: PCT/US21/64937

§ 371 Date: Jun. 13, 2023

Related U.S. Application Data
(60) Provisional application No. 63/129,311, filed on Dec. 22, 2020.

(51) Int. Cl. C12N 9/48; A61K 38/48

(52) U.S. Cl. CPC C12N 9/485; A61K 38/4813; C12Y 304/17023

(57) ABSTRACT

The disclosure is directed to methods of inhibiting coronavirus infection using a hydrocarbon stapled peptide which is a peptidomimetic of the human angiotensin-converting enzyme 2 (hACE2). The hydrocarbon stapled peptide binds to the receptor binding domain (RBD) of a coronavirus spike protein (S) and inhibits binding of the coronavirus to hACE2 expressed on the surface of host cells.

### SEQUENCE LISTING

The text of the computer readable sequence listing filed herewith, titled “39077-601 SEQUENCE LISTING ST25”, created Dec. 21, 2021, having a file size of 35,608 bytes, is hereby incorporated by reference in its entirety.

### FIELD

The disclosure is directed to methods of inhibiting coronavirus infection using a hydrocarbon stapled peptide which is a peptidomimetic of the human angiotensin-converting enzyme 2 (hACE2). The hydrocarbon stapled peptide binds to the receptor binding domain (RBD) of a coronavirus spike protein (S) and inhibits binding of the coronavirus to hACE2 expressed on the surface of host cells.

### BACKGROUND

According to the U.S. Department of Health and Human Services Centers for Disease Control and Prevention (CDC), Chinese authorities identified an outbreak caused by a novel coronavirus termed SARS-CoV-2. The virus can cause mild to severe respiratory illness, known as Coronavirus Disease 2019 (COVID-19), formerly called 2019-nCoV (van Dorp L et al., Infec Genet Evol, 83 :104351 (2020)). The outbreak began in Wuhan, Hubei Province, China and has spread to a growing number of countries worldwide, including the United States. On Mar. 11, 2020, the World Health Organization declared COVID-19 a pandemic. SARS-CoV-2 is different from six other identified human coronaviruses, including those that have caused previous outbreaks of Severe Acute Respiratory Syndrome (SARS) and Middle East Respiratory Syndrome (MERS). The U.S. Food and Drug Administration (FDA) has not yet approved a drug specifically indicated for the treatment of COVID-19 or a COVID-19 vaccine.

SARS-CoV-2 enters cells via the receptor binding domain (RBD) of its spike protein (S) binding to human angiotensin-converting enzyme 2 (hACE2) protein on host cells. hACE2 is commonly expressed on the surface of cells in the lungs, arteries, heart, kidney, and intestines. As such, the protein-protein interaction (PPI) between RBD and hACE2 has been explored as a therapeutic target to directly inhibit viral infection. Other approaches have been used to inhibit the RBD/hACE2 PPI, such as soluble ACE2 protein and native ACE2 peptides. However, these approaches have critical limitations, such as poor bioavailability due to rapid clearance, proteolytic degradation, and poor target cell delivery.

Thus, there remains a need for methods and compositions that inhibit and treat coronavirus infection.

### BRIEF SUMMARY OF THE INVENTION

The disclosure provides a method of inhibiting binding of a coronavirus to human angiotensin-converting enzyme 2 (hACE2) expressed on the surface of a cell, which comprises contacting one or more cells expressing an hACE2, with a hydrocarbon stapled peptide, whereby the hydrocarbon stapled peptide binds to the receptor binding domain (RBD) of a coronavirus spike protein (S) and inhibits binding of the coronavirus (e.g., SARS-CoV-2) to hACE2.

The disclosure also provides a method of treating a coronavirus infection in a subject, which comprises administering to the subject a hydrocarbon stapled peptide, whereby the hydrocarbon stapled peptide binds to the receptor binding domain (RBD) of a coronavirus spike protein (S) and inhibits binding of the coronavirus to hACE2 expressed by cells in the subject, thereby treating the coronavirus infection in the subject.

The disclosure further provides the use of a hydrocarbon stapled peptide for the treatment of a coronavirus infection in a subject.

The disclosure also provides a composition comprising a hydrocarbon stapled peptide and a pharmaceutically acceptable carrier, wherein the hydrocarbon stapled peptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or active variants thereof with modified sequences and/or alternative stapling strategies.

### BRIEF DESCRIPTION OF THE DRAWING(S)

FIG. 1A is a schematic diagram illustrating the structural interaction between the spike SARS-CoV-2 receptor binding domain (RBD) and the al helix of the ACE2 protein.

FIG. 1B is a helical wheel diagram of the primary sequence of 23 residues from the amphipathic al helix of the ACE2 protein (SEQ ID NO: 26). The SARS-CV-2/hACE2 protein-protein interaction (PPI) is mediated by the polar face of the helix (indicated by the curved dotted red line).

FIG. 2A is a chart showing peptide sequences and residues used for the synthesis of stapled alpha-helical peptides (SAHPs). Those residues critical for the binding to the SARS-CoV-2 RBD highlighted in red. Staples are bridged by i,(i+4) staples shown in blue linking two artificial amino acid residues. SAHPs ACE2-11 and 12 represent hydrocarbon “stitched” peptides. Certain exemplary point mutations predicted to increase target binding affinity are highlighted in green.

FIG. 2B is a representative graph of circular dichroism measurements showing that the native (unstapled) ACE2 al helix exists as a random coil at temperatures ranging from 5-95° C.

FIG. 2C is a representative graph of circular dichroism measurements showing that SAHPs, as represented by SAHP ACE2-7, are much more α-helical than the native, unstapled, ACE 2 α1 helix.

FIGS. 2D-F are graphs showing α-helicity of various stapled alpha-helical peptides (SAHPs) of the ACE2 α1 helix as measured by circular dichroism at a wide range of temperatures. SAHPs were dissolved at 25 μM in phosphate buffer at pH=7. Approximate physiologic temperature of 35° C. is highlighted by the dotted line.

FIG. 3 is a graph illustrating the trypsin-mediated proteolytic sensitivity of SAHP ACE2s near normal physiologic temperatures at 37° C. Double-stapled products are more proteolytically resistant than single-stapled moieties.

FIGS. 4A-B are graphs demonstrating prevention of SARS-CoV-2 infection in Vero E6 cells by ACE2 SAHPs. Various concentrations of stapled alpha-helical peptides (SAHPs) of the ACE2 α1 helix (100, 50, and 10 μM) were mixed with 400 plaque-forming units of SARS-CoV-2 live viruses for 1 hour and at 37° C. The mixture was then added to Vero E6 cells for an infection period of 72 hours. Cells were then fixed and stained with crystal violet to measure the live cell densities and compared to cells treated with virus alone and treated with a known COVID-19 neutralizing antibody (AM001414, 100 nM). To account for variability in the number of cells plated, cell density readouts at 10 μM for all SAHPs were treated as negative controls.

FIG. 5 is a graph demonstrating the dramatic decreases in viral replication and spike protein production in infected Vero E6 cells by compound ACE2-18. Various concentrations of stapled alpha-helical peptides (SAHPs) of the ACE2 al helix (100, 50, and 10 μM) were mixed with 500 plaque-forming units of SARS-CoV-2 live virus for 1 hour at 37° C. The mixture was then added to Vero E6 cells for 48 hours. Viral infection alone resulted in extensive spike protein expression within infected cells. This expression was quantified via cell fixation and spike protein immunohistochemistry (IHC). The percentage of cells positive for spike protein expression was then normalized to cells treated with a known COVID-19 neutralizing antibody (AM001414, 100 μM).

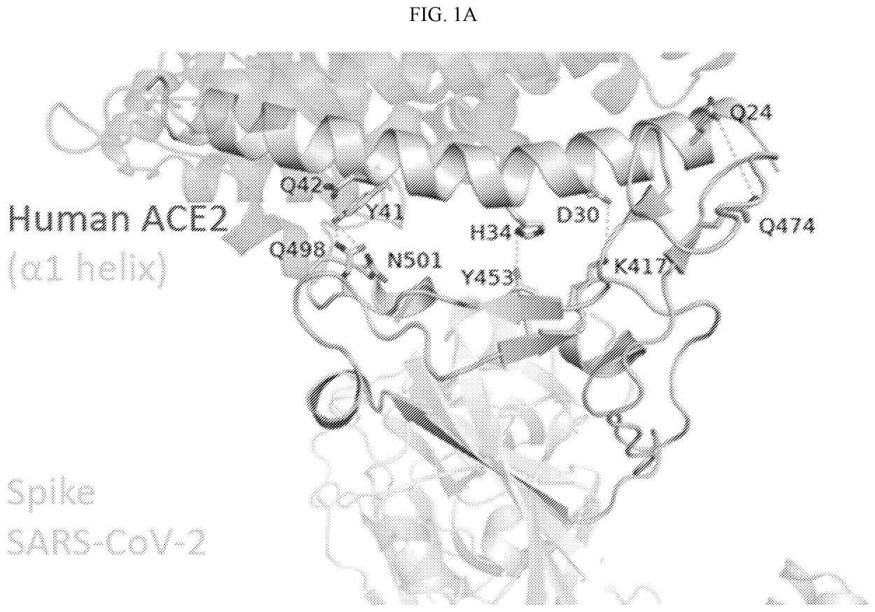

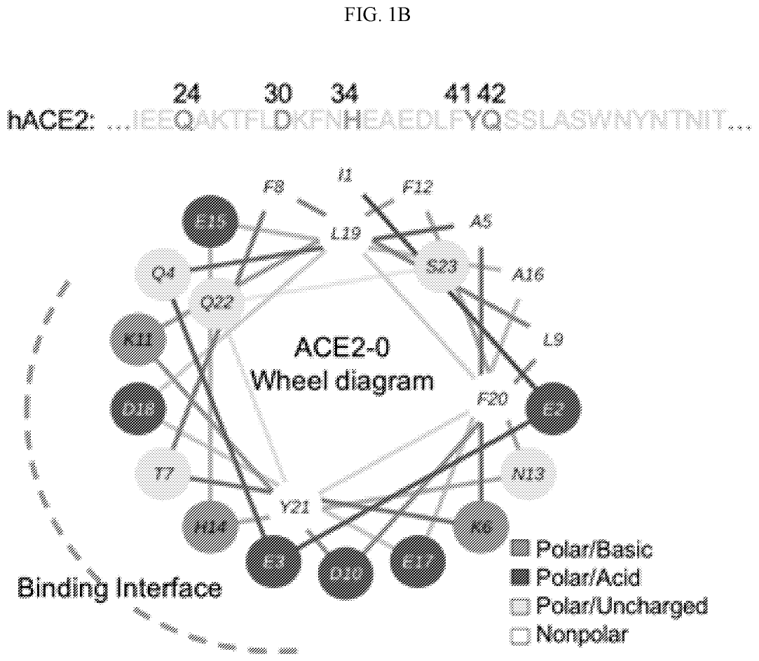

### DETAILED DESCRIPTION

The present disclosure is predicated, at least in part, on the development of a hydrocarbon stapled peptide which was generated in the likeness of a key alpha-helical domain of the hACE2 protein responsible for binding the RBD of the SARS-CoV-2 spike protein. Both single- and double-stapled/stitched hydrocarbon peptides are proteolytically resistant and maintain their secondary shape at a wide range of biologically relevant temperatures. Such hydrocarbon stapled peptides may be used therapeutically to inhibit binding and entry of the SARS-CoV-2 virus through molecular mimicry.

### Definitions

To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” “contacting,” and “introducing,” are used interchangeably herein and refer to the placement of therapeutic agents into a subject by a method or route which results in at least partial localization a desired site. The therapeutic agents can be administered by any appropriate route which results in delivery to a desired location in the subject.

As used herein, the term “subject” refers to any mammal (e.g., human, non-human primate, rodent, feline, canine, bovine, porcine, equine, etc.). In some embodiments, the subject is at elevated risk for infection (e.g., by a coronavirus). In some embodiments, the subject may have a healthy or normal immune system. In some embodiments, the subject is one that has a greater than normal risk of being exposed to a pathogen (e.g., a coronavirus). In some embodiments, the subject is a soldier, an emergency responder or other subject that has a higher than normal risk of being exposed to a pathogen (e.g., a coronavirus).

As used herein, the terms “at risk for infection” and “at risk for disease” refer to a subject that is predisposed to experiencing a particular infection or disease (e.g., a coronavirus). This predisposition may be genetic, or due to other factors (e.g., immunosuppression, compromised immune system, immunodeficiency, environmental conditions, exposures to detrimental compounds present in the environment, etc.). Thus, it is not intended that embodiments of the present disclosure be limited to any particular risk (e.g., a subject may be “at risk for disease” simply by being exposed to and interacting with other people), nor is it intended that embodiments of the present disclosure be limited to any particular disease.

As used herein, the term “sample” is used in its broadest sense and encompass materials obtained from any source. In particular embodiments, the term “sample” is used to refer to materials obtained from a biological source (i.e., a “biological sample”), for example, obtained from mammals (including humans), and encompasses any fluids, solids, and tissues. In particular embodiments of the present disclosure, biological samples include blood and blood products such as whole blood, plasma, serum, dried blood spots, etc. In other embodiments, the sample is an oropharyngeal specimen, a nasopharyngeal specimen, sputum, saliva, endotracheal aspirate, or bronchoalveolar lavage. However, these examples are not to be construed as limiting the types of samples that find use with the present disclosure.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The term “amino acid” refers to natural amino acids, unnatural amino acids, and amino acid analogs, all in their D and L stereoisomers, unless otherwise indicated, if their structures allow such stereoisomeric forms.

Natural amino acids are amino acids that occur in nature without need for synthetic synthesis. Natural amino acids include the “proteinogenic amino acids” coded for in the human genetic code: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“Octan”), ornithine (“Orn”), pentylglycine (“pG” or “PGly”), pipecolic acid, thioproline (“ThioP” or “tPro”), homoLysine (“hLys”), homoArginine (“hArg”), (S)-N-Fmoc-2-(4′-pentenyl) alanine, Fmoc-2,2-bis(4-pentenyl) glycine. Other unnatural amino acids that may be employed are disclosed in, e.g., International Patent Application Publication WO 2018/106937.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, the term “mutant peptide” refers to a variant of a peptide having a distinct amino acid sequence from the most common variant occurring in nature, referred to as the “wild-type” sequence. A mutant peptide may be a subsequence of a mutant protein or polypeptide (e.g., a subsequence of a naturally-occurring protein that isn't the most common sequence in nature), or may be a peptide that is not a subsequence of a naturally occurring protein or polypeptide.

As used herein, the term “artificial peptide” refers to a peptide having a distinct amino acid sequence from those found in natural peptides and/or proteins. An artificial protein is not a subsequence of a naturally occurring protein, either the wild-type (i.e., most abundant) or mutant versions thereof An “artificial peptide,” as used herein, may be produced or synthesized by any suitable method (e.g., recombinant expression, chemical synthesis, enzymatic synthesis, etc.).

The terms “polypeptide” and “protein” are used interchangeably herein and refer to a polymeric form of amino acids of any length, which can include coded and non-coded amino acids, chemically or biochemically modified or derivatized amino acids, and polypeptides having modified peptide backbones.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another: (1) Alanine (A) and Glycine (G); (2) Aspartic acid (D) and Glutamic acid (E); (3) Asparagine (N) and Glutamine (Q); (4) Arginine (R) and Lysine (K); (5) Isoleucine (I), Leucine (L), Methionine (M), and Valine (V); (6) Phenylalanine (F), Tyrosine (Y), and Tryptophan (W); (7) Serine (S) and Threonine (T); and (8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural reside. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

“Peptide stapling” is a term coined from a synthetic methodology in which two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed.37:3281, 1994). As used herein, the term “peptide stapling” includes the joining of two (e.g., at least one pair of) double bond-containing side-chains, triple bond- containing side-chains, or double bond-containing and triple bond-containing side chain, which may be present in a polypeptide chain, using any number of reaction conditions and/or catalysts to facilitate such a reaction, to provide a singly “stapled” polypeptide. The term “multiply stapled” polypeptides refers to those polypeptides containing more than one individual staple, and may contain two, three, or more independent staples of various spacing. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced. Hydrocarbon stapled polypeptides include one or more tethers (linkages) between two non-natural amino acids, which tether significantly enhances the α-helical secondary structure of the polypeptide. Generally, the tether extends across the length of one or two helical turns (i.e., about 3.4 or about 7 amino acids). Exemplary stapled peptides include those described in U.S. Pat. No. 10/259,848, International Patent Application Nos. WO2012/142604 and WO2018106937, each incorporated herein by reference in its entirety.

The terms “peptidomimetic” and “peptide mimic” may be used interchangeably herein to refer to compounds whose essential elements mimic a natural peptide or protein in 3D space and which retain the ability to interact with a biological target and produce the same biological effect. Peptidomimetics and their potential therapeutic applications are further described in, for example, Vagner et al., Curr Opin Chem Biol., 12(3): 292-296 (2008); and Mabonga, L., Kappo, A. P., Int J Pept Res Ther, 26, 225-241 (2020).

The term “polymersome,” as used herein, refers to a type of artificial vesicle that encloses a solution. The solutions within and outside the polymersome may be the same or different. Polymersomes are made using amphiphilic synthetic block copolymers to form the vesicle membrane. The copolymer may be, for instance, a diblock or a triblock copolymer. The polymersome membrane provides a physical barrier that isolates the encapsulated material from external materials, such as those found in biological systems. Polymersomes can be generated from double emulsions by known techniques, see Lorenceau et al., 2005, Langmuir 21(20):9183-6, incorporated herein by reference in its entirety.

The term “recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, the artificial combination may be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may comprise a naturally occurring amino acid sequence.

As used herein, the terms “treat,” “treating,” and the like means a slowing, stopping, or reversing the progression of a disease or disorder. As such, “treating” means an application or administration of the methods or agents described herein to a subject, where the subject has a disease or a symptom of a disease, where the purpose is to cure, heal, alleviate, relieve, alter, remedy, ameliorate, improve or affect the disease or symptoms of the disease.

### Coronaviruses

The disclosure provides a method of inhibiting binding of a coronavirus to hACE2 expressed on the surface of a cell. Coronaviruses are a large family of viruses that are common in humans and many different species of animals, including camels, cattle, cats, and bats. Coronaviruses are named for the crown-like spikes on their surface. There are four main sub-groupings of coronaviruses, known as alpha, beta, gamma, and delta. Human coronaviruses were first identified in the mid-1960s. Seven coronaviruses have been identified that can infect humans: 229E (alphacoronavirus); NL63 (alphacoronavirus); 0C43 (betacoronavirus); HKU1 (betacoronavirus); MERS-CoV (the betacoronavirus that causes Middle East Respiratory Syndrome, or MERS); SARS-CoV (the betacoronavirus that causes severe acute respiratory syndrome, or SARS); and SARS-CoV-2 (the novel coronavirus that causes coronavirus disease 2019, or COVID-19). Rarely, animal coronaviruses can infect and spread among humans, such as with MERS-CoV, SARS-CoV, and SARS-CoV-2 (COVID-19). The SARS-CoV-2 virus is a betacoronavirus, like MERS-CoV and SARS-CoV. All three of these viruses originated in bats. MERS-CoV and SARS-CoV have been known to cause severe illness in people. The complete clinical picture with regard to COVID-19 is not fully understood. Reported illnesses have ranged from mild to severe, including illness resulting in death. While recent data suggests that most COVID-19 illness is mild, a report out of China suggests serious illness occurs in 16% of cases. Older people and people with certain underlying health conditions like heart disease, lung disease and diabetes, for example, seem to be at greater risk of serious illness.

In some embodiments, the method inhibits binding of SARS-CoV-2 to hACE2 expressed on the surface of cells (e.g., human host cells). SARS-CoV-2 is a monopartite, single-stranded, and positive-sense RNA virus with a genome size of 29,903 nucleotides, making it the second-largest known RNA genome. The virus genome consists of two untranslated regions (UTRs) at the 5′ and 3′ ends and 11 open reading frames (ORFs) that encode 27 proteins. The first ORF (ORF 1/ab) constitutes about two-thirds of the virus genome and encodes 16 non-structural proteins (NSPs), while the remaining third of the genome encodes four structural proteins and at least six accessory proteins. The structural proteins are spike glycoprotein (S), matrix protein (M), envelope protein (E), and nucleocapsid protein (N), while the accessory proteins are orf3a, orf6, orf7a, orf7b, orf8, and orf10 (Wu et al., Cell Host Microbe, 27: 325-328 (2020); Chan et al., Emerg. Microbes Infect., 9: 221-236 (2020); Chen et al., Lancet, 395: 507-513 (2020); and Ceraolo, C.; Giorgi, F. M, J Med. Virol., 92, 522-528 (2020)). Of the NSPs, (1) NSP1 suppresses the antiviral host response, (2) NSP3 is a papain-like protease, (3) NSPS is a 3CLpro (3C-like protease domain), (4) NSP7 makes a complex with NSP8 to form a primase, (5) NSP9 is responsible for RNA/DNA binding activity, (6) NSP12 is an RNA-dependent RNA polymerase (RdRp), (7) NSP13 is confirmed as a helicase, (8) NSP14 is a 3′-5′ exonuclease (ExoN), (9) NSP15 is a poly(U)-specific endoribonuclease (XendoU). The remaining NSPs are involved in transcription and replication of the viral genome (Chan et al., Emerg. Microbes Infect., 9: 221-236 (2020); and Krichel et al., Biochem. J., 477: 1009-1019 (2019)). The genome of SARS-CoV-2 has been sequenced, and the nucleic acid sequences and amino acid sequences of all SARS-CoV-2 proteins are publicly available (see NCBI Reference Sequence: NC_045512.2).

The spike (S) protein (˜150 kDa) is heavily N-linked glycosylated and utilizes an N-terminal signal sequence to gain access to the endoplasmic reticulum (ER). The amino acid sequence of the SARS-CoV-2 spike protein has been deposited with the National Center for Biotechnology Information (NCBI) under Accession No. QHD43416. Homotrimers of the virus-encoding S protein make up the distinctive spike structure on the surface of the virus. In many, but not all, coronaviruses, the S protein is cleaved by a host cell furin-like protease into two separate polypeptides known as S1 and S2. S1 makes up the large receptor binding domain (RBD) of the S protein while S2 forms the stalk of the spike molecule. The trimeric S glycoprotein mediates attachment of the coronavirus virion to the host cell by interactions between the S protein and its receptor, human ACE2 (referred to herein as “hACE2” or “ACE2”) (Zhou et al., Nature 579: 270-273, doi:10.1038/s41586-020-2012-7 (2020); Hoffmann et al., Cell, S0092-8674(0020)30229-30224, doi:10.1016/j.cell.2020.02.052 (2020) doi:10.1016/j.cell.2020.02.052 (2020). Binding with ACE2 triggers a cascade of cell membrane fusion events for viral entry. The high-resolution structure of SARSCoV-2 RBD bound to the N-terminal peptidase domain of ACE2 has recently been determined, and the overall ACE2-binding mechanism is virtually the same between SARS-CoV-2 and SARS-CoV RBDs, indicating convergent ACE2-binding evolution between these two viruses (Gui et al., CellRes 27, 119-129, doi:10.1038/cr.2016.152 (2017); Song et al., PLoS Pathog 14, e1007236-e1007236, doi:10.1371/journal.ppat.1007236 (2018); Yuan et al., Nat Commun 8, 15092-15092, doi:10.1038/ncomms15092 (2017); and Wan et al., J Virol, JVI.00127-00120, doi:10.1128/JVI.00127-20 (2020)). This suggests that disruption of the RBD and ACE2 interaction would block SARS-CoV-2 entry into the target cell. Indeed, a few such disruptive agents targeting ACE2 have been shown to inhibit SARS-CoV infection (Kruse, R. L., F1000Res, 9: 72-72; doi:10.12688/f1000research.22211.2 (2020); and Li et al., Nature 426, 450-454; doi:10.1038/nature02145 (2003)).

### Hydrocarbon Stapled Peptides

Protein-protein interactions (PPI) between host and virus are valuable targets to inhibit viral replication. However, the large and flat interacting surfaces of PPI often preclude the use of small molecules as drugs to disrupt these PPI. Larger biologics, such as hydrocarbon-stapled peptide mimetics, are, on the other hand, promising tools for disrupting intracellular PPI that were previously intractable (Ali et al., Comput Struct Biotechnol J 2019, 17, 263-281; Iegre et al., Chem Sci 2018, 9 (20), 4638-4643; Moiola et al., Molecules 2019, 24 (20); Muppidi et al., Bioorg Med Chem Lett 2014, 24 (7), 1748-51; and Curreli et al., bioRxiv 2020.08.25.266437; doi: doi.org/10.1101/2020.08.25.266437). Stapled peptides are typically derived from the α-helix of the binding interface, and they are locked into bioactive conformations through the use of chemical linkers. Stapling enhances α-helicity of unstructured short peptides in solution, improves resistance against proteolytic digestion, as well as potency, and often improves cell penetration (Charoenpattarapreeda et al., Chem Commun (Camb) 2019, 55 (55), 7914-7917; Cowell et al., Cancer Growth Metastasis 2017, 10, 1179064417713197; Cromm et al., ACS Chem Biol 2015, 10 (6), 1362-75; Dietrich et al., Cell Chem Biol 2017, 24 (8), 958-968 e5; Dougherty, J Med Chem 2019, 62 (22), 10098-10107; Gaillard et al., Antimicrob Agents Chemother 2017, 61 (4); and Verdine, G. L.; Hilinski, G. J., Methods Enzymol 2012, 503, 3-33).

The hydrocarbon stapled peptide employed in the methods provided here desirably is a peptidomimetic of hACE2. ACE2 is a membrane-associated aminopeptidase expressed in vascular endothelia, renal and cardiovascular tissue, and epithelia of the small intestine and testes (Donoghue et al., Circ. Res. 87:el-e9 (2000); Hamming et al., J. Pathol. 203:631-637 (2004); and Harmer et al., FEBS Lett. 532:107-110 (2002)). A region of the extracellular portion of ACE2 that includes the first α-helix and lysine 353 and proximal residues of the N terminus of β-sheet 5 interacts with high affinity to the receptor binding domain of the SARS-CoV S glycoprotein (Li et al., EMBO J. 24:1634-1643 (2005)). In some embodiments, the hydrocarbon stapled peptide mimics the alpha-helical domain of the ACE2 receptor. In some embodiments, the stapled peptide is based on an alpha-helical domain of hACE2 comprising IEEQAKTFLDKFNHEAEDLFYQS (SEQ ID NO: 1) and comprises 1 to 10 (e.g., 1, 2, 3, 4, 5, 6, 7, 8) amino acid substitutions, deletions and/or insertions as compared to SEQ ID NO: 1. For example, the stapled peptide can include at least two (e.g., 2, 3, 4, 5, 6, 7, 8) amino acid substitutions of SEQ ID NO: 1, wherein the substituted amino acids are separated by two, three, or six amino acids, and wherein the substituted amino acids are non-natural amino acids with olefinic side chains. In some embodiments, the stapled peptide is based on an alpha-helical domain of hACE2 comprising STIEEQAKTFLDKFNHEAEDLFYQSSL (SEQ ID NO: 14) and comprises 1 to 10 (e.g., 1, 2, 3, 4, 5, 6, 7, 8) amino acid substitutions, deletions and/or insertions as compared to SEQ ID NO: 14. For example, the stapled peptide can include at least two (e.g., 2, 3, 4, 5, 6, 7, 8) amino acid substitutions of SEQ ID NO: 14, wherein the substituted amino acids are separated by two, three, or six amino acids, and wherein the substituted amino acids are non-natural amino acids with olefinic side chains. In some embodiments, the stapled peptide comprises a single pair of substituted amino acids separated by two, three, or six amino acids that facilitate the formation of a staple linkage. In some embodiments, the stapled peptide comprises two pairs of substituted amino acids respectively separated by two, three, or six amino acids that facilitate the formation of two staple linkages. In some embodiments, stapled peptides may comprise additional amino acid substitutions or deletions, in addition to those that facilitate formation of the staple linkages.

In some embodiments, peptides comprises any combination of proteogenic, non-proteogenic, natural, and unnatural amino acids. There are many known non-natural or unnatural amino acids any of which may be included in the peptides of the present disclosure. Some examples of unnatural amino acids are 4-hydroxyproline, desmosine, gamma-aminobutyric acid, beta-cyanoalanine, norvaline, 4-(E)-butenyl-4(R)-methyl-N- methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and/para-substituted phenylalanines (e.g., substituted with —C(═O)C₆H₅; —CF₃; —CN; —halo; —NO₂; CH₃), disubstituted phenylalanines, substituted tyrosines (e.g., further substituted with —C═O)C₆H₅; —CF₃; —CN; -halo; —NO₂; CH₃), and statine. Additionally, amino acids can be derivatized to include amino acid residues that are hydroxylated, phosphorylated, sulfonated, acylated, or glycosylated.

Hydrocarbon stapled peptides typically include one or more tethers (linkages) between two non-natural amino acids, which tether significantly enhances the α-helical secondary structure of the peptide. Generally, the tether extends across the length of one or two helical turns (i.e., about 3.4 or about 7 amino acids). Accordingly, amino acids positioned at i and i+3; i and i+4; or i and i+7 are ideal candidates for chemical modification and cross-linking. Thus, for example, where a peptide has the sequence . . . X1, X2, X3, X4, X5, X6, X7, X8, X9 . . . , cross-links between X1 and X4, or between X1 and X5, or between X1 and X8 are useful hydrocarbon stapled forms of that peptide, as are cross-links between X2 and X5, or between X2 and X6, or between X2 and X9, etc. The use of multiple cross-links (e.g., 2, 3, 4, or more) is also contemplated. The use of multiple cross-links is very effective at stabilizing and optimizing the peptide, especially with increasing peptide length. Thus, the disclosure encompasses the incorporation of more than one cross-link within the polypeptide sequence to either further stabilize the sequence or facilitate the structural stabilization, proteolytic resistance, acid stability, thermal stability, cellular permeability, and/or biological activity enhancement of longer polypeptide stretches. Additional description regarding making and use of hydrocarbon stapled polypeptides can be found, e.g., in U.S. Patent Publication Nos. 2012/0172285, 2010/0286057, and 2005/0250680s. In some embodiments, exemplary peptide sequences are provided herein with pairs “X” amino acids at locations of linkages (SEQ ID NOS: 2-13 and 15-25).

In one aspect, a hydrocarbon stapled polypeptide has the formula (I),

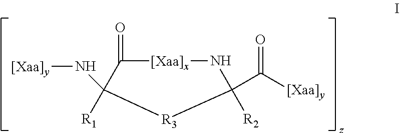

wherein:

each R₁ and R₂ are independently H or a C₁ to C₁₀ alkyl, alkenyl, alkynyl, arylalkyl, cycloalkylalkyl, heteroarylalkyl, or heterocyclylalkyl;

R₃ is alkyl, alkenyl, alkynyl; [R₄—K—R₄]ₙ; each of which is substituted with 0-6 R₅;

R₄ is alkyl, alkenyl, or alkynyl;

R₅ is halo, alkyl, OR₆, N(R₆)₂, SR₆, SOR₆, SO₂R₆, CO₂R₆, R₆, a fluorescent moiety, or a radioisotope;

K is O, S, SO, SO₂, CO, CO₂, CONR₆, or

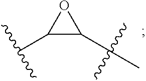 [SMILES: *C1OC1*]

R₆ is H, alkyl, or a therapeutic agent;

n is an integer from 1-4;

x is an integer from 2-10;

each y is independently an integer from 0-100;

z is an integer from 1-10 (e.g., 1, 2, 3, 4, 5, 6, 7, 8, 9, 10);

and each Xaa is independently an amino acid. The stapled polypeptides can include an amino acid sequence of an hACE2 alpha-helical domain as described herein.

In some embodiments, the tether includes an alkyl, alkenyl, or alkynyl moiety (e.g., C₅, C₈, or C₁₁ alkyl, a C₅, C₈, or C₁₁ alkenyl, or C₅, C₈, or C₁₁ alkynyl). In some embodiments, the tethered amino acid is alpha disubstituted (e.g., C₁-C₃ or methyl).

In some instances, x is 2, 3, or 6. In some instances, each y is independently an integer between 1 and 15, or 3 and 15. In some instances, R₁ and R₂ are each independently H or C₁-C₆ alkyl. In some instances, R₁ and R₂ are each independently C₁-C₃ alkyl. In some instances, at least one of R₁ and R₂ are methyl. For example, R₁ and R₂ can both be methyl. In some instances, R₃ is alkyl (e.g., C₈ alkyl) and x is 3. In some instances, R₃ is C₁₁ alkyl and x is 6. In some instances, R₃ is alkenyl (e.g., Cs alkenyl) and x is 3. In some instances, x is 6 and R₃ is C₁₁ alkenyl. In some instances, R3 is a straight chain alkyl, alkenyl, or alkynyl. In some instances, R₃ is —CH₂—CH₂—CH₂—CH═CH—CH₂—CH₂—CH₂—.

In another aspect, the two alpha, alpha disubstituted stereocenters are both in the R configuration or S configuration (e.g., i, i+4 cross-link), or one stereocenter is R and the other is S (e.g., i, i+7 cross-link). Thus, where formula I is depicted as:

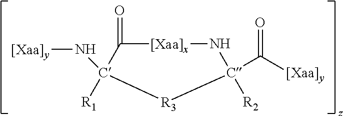

the C′ and C″ disubstituted stereocenters can both be in the R configuration or they can both be in the S configuration, e.g., when x is 3. When x is 6, the C′ disubstituted stereocenter is in the R configuration and the C″ disubstituted stereocenter is in the S configuration. The R₃ double bond can be in the E or Z stereochemical configuration.

In some instances, R₃ is [R₄—K—R₄]ₙ; and R₄ is a straight chain alkyl, alkenyl, or alkynyl.

In some embodiments, the disclosure features internally cross-linked (“stapled” or “stitched”) peptide variants of the al helical domain of hACE2, which has the amino acid sequence IEEQAKTFLDKFNHEAEDLFYQS (SEQ ID NO: 1) or STIEEQAKTFLDKFNHEAEDLFYQSSL (SEQ ID NO: 14), wherein the side chains of two amino acids separated by two, three, or six amino acids are replaced by an internal staple; the side chains of three amino acids are replaced by an internal stitch; the side chains of four amino acids are replaced by two internal staples, or the side chains of five amino acids are replaced by the combination of an internal staple and an internal stitch. In certain instances, the amino acids at one or more of positions 4, 10, 14, 21, and 22 of SEQ ID NO: 1 are not replaced with a staple or stitch. In other embodiments, the amino acids at one or more pairs of positions selected from 1 and 5, 8 and 12, 9 and 13, 12 and 16, 16 and 20 of SEQ ID NO: 1 are replaced with a staple or stitch. In certain embodiments, the amino acids at one or more pairs of positions selected from 1 and 5, 2 and 6, 8 and 12, 9 and 13, 12 and 16, and 16 and 20 of SEQ ID NO: 14 are replaced with a staple or stitch. As such, the hydrocarbon stapled peptide may comprise a single staple or multiple staples (e.g., 2, 3, 4, 5 or more staples, i.e., “stitched” as defined above). In some embodiments, the hydrocarbon stapled peptide is single- or double-stapled. The stapled/stitched peptide can be 15, 16, 17, 18, 19, 20, 21, 22, 23, 24, or 25 amino acids in length. Exemplary hydrocarbon stapled peptides that may be used in the methods described herein comprise an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25. In some embodiments, a hydrocarbon stapled peptide is provided having 70% or more (e.g., 70%, 75%, 80%, 85%, 90%, 95%, 100%) sequence identity to one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO:

25.

Embodiments herein are not limited to hydrocarbon tethers; other tethers are also employed in the peptides described herein. For example, in some embodiments, other tethers include one or more of an ether, thioether, ester, amine, or amide, or triazole moiety. In some cases, a naturally occurring amino acid side chain is incorporated into the tether. For example, a tether is coupled with a functional group such as the hydroxyl in serine, the thiol in cysteine, the primary amine in lysine, the acid in aspartate or glutamate, or the amide in asparagine or glutamine. Accordingly, in some embodiments, a tether is created using naturally occurring amino acids rather than using a tether that is made by coupling two non-naturally occurring amino acids. It is also possible to use a single non-naturally occurring amino acid together with a naturally occurring amino acid. Triazole-containing (e.g., 1,4 triazole or 1,5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the hACE2 peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc.,113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U. S. A., 97:3803-3808 (2000); Double-click stapling: Lau et al., Chem. Sci., 5:1804-1809 (2014); Bis-lactam stapling: J. C. Phelan et al., J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3, i to i+4, and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

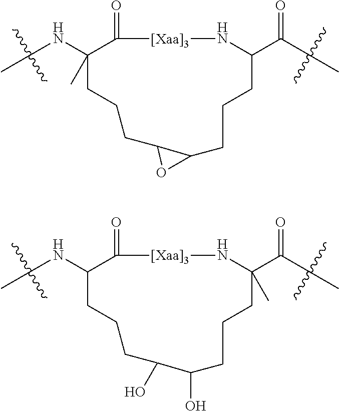

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the polypeptide or via the amino acid side chain. Other agents can be attached to the functionalized tether, e.g., an agent that facilitates entry of the polypeptide into cells.

In some instances, alpha disubstituted amino acids are used in the polypeptide to improve the stability of the alpha helical secondary structure. However, alpha disubstituted amino acids are not required, and instances using mono-alpha substituents (e.g., in the tethered amino acids) are also envisioned.

The stapled peptides can include a drug, a toxin, a derivative of polyethylene glycol; a second polypeptide; a carbohydrate, etc. Where a polymer or other agent is linked to the stapled polypeptide is can be desirable for the composition to be substantially homogeneous.

The addition of polyethelene glycol (PEG) molecules can improve the pharmacokinetic and pharmacodynamic properties of the stapled peptide. For example, PEGylation can reduce renal clearance and can result in a more stable plasma concentration. PEG is a water-soluble polymer and can be represented as linked to the polypeptide as formula:

XO—(CH2CH2O)n—CH2CH2—Y

where n is 2 to 10,000 and X is H or a terminal modification, e.g., a C1-4 alkyl; and Y is an amide, carbamate or urea linkage to an amine group (including but not limited to, the epsilon amine of lysine or the N-terminus) of the peptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain embodiments, macromolecular polymer (e.g., PEG) is attached to a stapled peptide described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

In some embodiments, the stapled peptides can also be modified, e.g., to further facilitate cellular uptake or increase in vivo stability. For example, acylating or PEGylating a peptidomimetic macrocycle facilitates cellular uptake, increases bioavailability, increases blood circulation, alters pharmacokinetics, decreases immunogenicity and/or decreases the needed frequency of administration.

In some embodiments, the stapled peptides disclosed herein have an enhanced ability to penetrate cell membranes (e.g., relative to non-stapled peptides).

Methods of synthesizing the compounds of the described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this invention can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH₂ protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech.

Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂-CH₂); a thioamide bond (C(S)—NH); a trans-olefin bond (CH═CH); a fluoro substituted trans-olefin bond (CF═CH); a ketomethylene bond (C(O)-CHR) or CHR-C(O) wherein R is H or CH₃; and a fluoro-ketomethylene bond (C(O)-CFR or CFR-C(O) wherein R is H or F or CH₃.

The polypeptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C₁-C₂₀ straight or branched alkyl groups); fatty acid radicals; and combinations thereof. α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). For peptides where an i linked to 1+7 staple is used (two turns of the helix stabilized) either: a) one S5 amino acid and one R8 is used; or b) one S8 amino acid and one R5 amino acid is used. R8 is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids Fmoc-S₅-OH, Fmoc-R₈-OH , Fmoc-R₈-OH, Fmoc-S₈-OH and Fmoc-R₅-OH), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, Calif.). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stapling, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some embodiments, the peptides are substantially free of non-stapled peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific embodiment, a 50%/50% DMSO/NMP solution is used. The solution may be incubated for a period of 1, 6, 12 or 24 hours, following which the resin may be washed, for example with dichloromethane or NMP. In one embodiment, the resin is washed with NMP. Shaking and bubbling an inert gas into the solution may be performed.

Whatever hydrocarbon stapled peptide is used, the hydrocarbon stapled peptide desirably binds to the receptor binding domain (RBD) of a coronavirus spike protein (S) and inhibits binding of the coronavirus to hACE2 expressed on the surface of a cell. Binding of a coronavirus to hACE2 is “inhibited” when the ability of a coronavirus (e.g., SARS-CoV-2) to bind hACE2 is impeded or disrupted, in whole or in part. Ideally, the hydrocarbon stapled peptide completely blocks binding of a coronavirus to hACE2 expressed on the surface of cells (e.g., human host cells). The degree of inhibition may be partially complete (e.g., 10% or more, 25% or more, 50% or more, or 75% or more), substantially complete (e.g., 85% or more, 90% or more, or 95% or more), or fully complete (e.g., 98% or more, or 99% or more).

In some embodiments, the stapled peptides that inhibit bind to the RBD of a coronavirus spike protein described herein may be modified to enhance biostability and/or biocompatibility, to extend the serum half-life, to prevent/inhibit/reduce clearance (e.g., by the kidneys), and/or to enhance therapeutic efficacy. Modification may include substitution/deletion/addition of amino acids from the sequences herein. In some embodiments, the stapled peptide comprises one or more substitutions (e.g., 1, 2, 3, 4, 5, 6, 7, 8, 9, 10, 11, 12, 13, 14, 15, 16, 17, 18, 19, 20, or ranges therebetween) relative to a peptide sequence provided herein. In some embodiments, a hydrocarbon stapled peptide that binds to the RBD of a coronavirus spike protein comprises a truncation (or deletion) relative to a peptide sequence described herein. In some embodiments, a truncation (or deletion) is at the C-terminus, N-terminus, or internally. In some embodiments, a hydrocarbon stapled peptide that binds to the RBD of a coronavirus spike protein comprises at least 70% (e.g., 70%, 75%, 80%, 85%, 90%, 95%, 99%, 100%) sequence identity with all or a portion of a peptide sequence provided herein. In some embodiments, a hydrocarbon stapled peptide that binds to the RBD of a coronavirus spike protein comprises at least 70% (e.g., 70%, 75%, 80%, 85%, 90%, 95%, 99%, 100%) sequence similarity (e.g., conservative or semiconservative) with all or a portion of a peptide sequence provided herein. In some embodiments, a hydrocarbon stapled peptide that binds to the RBD of a coronavirus spike protein comprises one or more modified or unnatural amino acids. In some embodiments, a hydrocarbon stapled peptide that binds to the RBD of a coronavirus spike protein is PEGylated, methylated, biotinylated, sumolyated, or otherwise modified.

A naturally occurring amino acid within the peptide sequences herein may be replaced with, for example, a non-naturally occurring amino acid such as, for example, norleucine, omithine, norvaline, homoserine, and other amino acid residue analogues such as those described in Ellman et al., Meth. Enzym., 1991, 202, 301-336. To generate such non-naturally occurring amino acid residues, the procedures of Noren et al., Science, 1989, 244, 182 and Ellman et al., supra, can be used. Other suitable methods are described in White et al., Methods, 2013, 60, 70-74; Gentilucci et al., Curr Pharm Des 2010, 16, 3195-3203; Hodgson & Sanderson, Chem Soc Rev 2004, 33, 422-430 and Krebs et al., Chemistry 2004, 10:544-553.

In some embodiments, the peptides described herein may be modified. Some modifications may increase the stability and activity of a peptide to enable reduced dosing level or frequency, as well as enable alternative routes of administration. Examples of modifications of peptides that may increase stability, activity, specificity, and/or efficacy include: replacing labile amino acids with ones that increase stability and improve activity (e.g., repalcing lysines/argininesthat are recognized by trypsin with glutamine); repolacing one or more L-amino acids with D-amino acids (Powell et al. Pharm. Res., 1993, 10, 1268-1273); reducing the size of the peptide bemoving non-essential sequences or individual residues (Harris, Gut, 1994, 35(3 Suppl), S1-4); PEGylating; C-terminal amidation or N-terminal acetylation (Brinckerhoff et al. Int'l J. Cancer, 1999, 83, 326-334), or N-pyroglutamylation (Green et al. J. Endocrinol., 2004, 180, 379-388); conjugation of various fatty acids ranging from 4-18 chain length as described in, for example, DasGupta et al., Biol. Pharma. Bull., 2002, 25, 29-36; adding biodegradable modifications (e.g., polymers of N-acetylneuraminic adid (poysialic acids)) as described in, for example, Georgiadis et al., Cell. Mol. Life Sci., 2000, 57, 1964-1969.

Other modifications may further include conjugation of the stapled peptide with a biologically active agent, label or diagnostic agent (e.g., as at the N-terminus, C-terminus, on an amino acid side chain, or at one or more modified or unmodified stapled sites, etc.). Such modification may be useful in delivery of the peptide to a cell, tissue, or organ. Such modifications may allow for targeting to a particular type of cell or tissue. Conjugation of an agent (e.g., a label, a diagnostic agent, a biologically active agent) to the peptide may be achieved in a variety of different ways. The agent may be covalently conjugated directly or indirectly, covalently, or non-covalently. Conjugation may be by amide linkages, ester linkages, disulfide linkages, carbon-carbon bonds, carbamate, carbonate, urea, hydrazide, and the like. In some embodiments, the conjugation is cleavable under physiological conditions (e.g., enzymatically cleavable, cleavable with a high or low pH, with heat, light, ultrasound, x-ray, etc.). However, in some embodiments, the bond is not cleavable.

### Compositions

For delivery to cells and areas of viral infection, the hydrocarbon stapled peptide described herein desirably is present in a composition which comprises a carrier, preferably a pharmaceutically (e.g., physiologically acceptable) carrier. Any suitable carrier can be used within the context of the disclosure, and such carriers are well known in the art. The choice of carrier will be determined, in part, by the particular site to which the composition is to be administered and the particular method used to administer the composition.

In some embodiments, the disclosure provides a composition comprising a hydrocarbon stapled peptide and a pharmaceutically acceptable carrier, wherein the hydrocarbon stapled peptide comprises the amino acid sequence of Seq ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25. In some embodiments, a hydrocarbon stapled peptide is provided having 70% or more (e.g., 70%, 75%, 80%, 85%, 90%, 95%, 100%) sequence identity to one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

In some embodiments, the hydrocarbon stapled peptide is encapsulated in a polymersome. Polymersomes are, like liposomes, vesicles having a membrane which encapsulates an interior solution from an exterior environment. However, polymersomes are formed using amphiphilic non-lipid polymers and the membrane may be a bilayer membrane or a single layer, as in a micelle.

Polymersome membranes commonly are generated using amphiphilic block copolymers. The copolymer may be, for instance, a diblock or a triblock copolymer. Polymersomes employed in the methods described herein may comprise amphiphilic disulfide block co-polymers. Amphiphilic disulfide block co-polymers have a disulfide group linking two block copolymers such that the block co-polymer is hydrolyzed in reducing environments. Thus, the block copolymers are reduction-sensitive such that when the polymersomes are taken up by the cell, they are disrupted in the endosome.

The amphiphilic block copolymers comprise at least one of a hydrophilic block and a hydrophobic block. The hydrophilic block may comprise poly(ethylene glycol), poly(ethylene oxide), poly(vinyl alcohol), poly(ethylene-co-vinyl alcohol), poly(N-vinyl pyrrolidone), poly(acrylic acid), poly(ethyloxazoline), poly(alkylacrylates), poly(acrylamides), poly(N-alkylacrylamides), or poly(N,N-dialkylacrylamides). The hydrophobic block may comprise poly(propylene sulfide), poly(propylene glycol), esterified poly(acrylic acid), esterified poly(glutamic acid), or esterified poly(aspartic acid).

In some embodiments, the amphiphilic disulfide block co-polymers are diblock copolymers comprising poly(ethylene glycol) (PEG) and poly(propylene sulfide) (PPS) with an intervening disulfide group separating the hydrophilic PEG from the hydrophobic PPS. The average molecular weight of the PEG may be between 750 and 1500 Da (e.g., 900-1300 Da, 1000-1500 Da, 1100-1400 Da). In some embodiments, the average molecular weight of the PEG is approximately 1000 Da. In some embodiments, the average molecule weight of the PEG is between 1200 and 1300 Da. The average molecular weight of the PPS may be between 3750 and 4500 Da (e.g., 3800-4500 Da, 3800-4200 Da, 4000-4200 Da). In some embodiments, the average molecular weight of the PPS is approximately 4000 Da.

The ratio of the molecular weights of the block polymers can influence the shape of the type of assembled vesicle, e.g. spherical polymersomes, bicontinuous nanospheres, long wormlike micelles (filomicelles), or spherical micelles (see, for example, Allen, S., et al., Journal of Controlled Release 262, 91-103 (2017)). Any ratio of molecular weights may be used that allow or facilitate formation of polymersomes.

The polymersomes desirably are on average about 100-150 nm. In some embodiments, the hydrodynamic radius of the polymersomes are between 50 and 150 nm. In some embodiments, the polymersomes are micelles with a hydrodynamic radius between 10 and 50 nm. The size of the polymersome may vary with the methods of making and the type and quantity of hydrocarbon stapled peptide encapsulated therein.

In other embodiments, the hydrocarbon stapled peptide may be packaged as a peptide amphiphile. The term “peptide amphiphile (PA)” as used herein, refers to peptide-based molecules that contain a hydrophilic peptide head group and one or more hydrophobic alkyl tails. Peptide amphiphiles can self-assemble into a variety of nanostructures, including, for example, micelles, vesicles, bilayers, and nanofibers. In some embodiments, PAs may contain a targeting or signaling epitope that allows the formed nanostructures to perform a biological function, either targeting or signaling, by interacting with living systems (Cui et al., Biopolymers. 94 (1): 1-18 (2010); Hendricks et al., Accounts of Chemical Research. 50 (10): 2440-2448 (2017)). In order for a fully functional PA to assemble into a well-defined supramolecular structure, four essential domains are required: (1) a hydrophobic tail to form hydrophobic interactions, (2) a peptide sequence that is able to form intermolecular hydrogen bonds, which determines the interfacial curvature of the self-assembled structure, (3) charged amino acids to promote solubility and (4) a functional peptide epitope (Dehsorkhi et al., J Pept Sci., 20(7): 453-467 (2014)). PAs are used in a variety of medical applications as nanocarriers, nanodrugs, and imaging agents. They are also frequently used in regenerative medicine to culture and deliver cells and growth factors (Pérez et al., Annals of Biomedical Engineering. 43 (3): 501-514 (2015)).

In some embodiments, the peptide segment of a PA may comprise the hydrocarbon stapled peptide described a linker peptide or structural peptide between the stapled peptide and the hydrophobic domain of the PA. In some embodiments, a structural peptide is a peptide that facilitates packing of the peptide amphiphiles into nanostructures. In some embodiments, noncovalent interactions (e.g., hydrogen bonds, beta sheet formation, van der Waals interactions, hydrophobic interactions, etc.) between structural peptides of adjacent PAs facilitate nanostructure formation and/or influence nanostructure size and/or shape.

In some embodiments, the hydrophobic segment of a PA comprises an acyl chain or lipid. For example, in some embodiments, the hydrophobic segment of a PA comprises a single, linear acyl chain of the formula: Cₙ₋₁H₂ₙ₋₁(O)—where n=6-22. In some embodiments, the hydrophobic segment is a lipid. In some embodiments, the lipid molecule is a fatty acid. In some embodiments, the fatty acid comprises 6-24 carbons. In some embodiments, lipids that find use in embodiments herein are fatty acids. In some embodiments, fatty acids in the compositions herein are a short chain fatty acid (carbon chain of <6 carbons (e.g., formic acid, acetic acid, propionic acid, butyric acid, isobutyric acid, valeric acid, isovaleric acid, etc.)), a medium chain fatty acid (carbon chain of 6-12 carbons (e.g., caproic acid, caprylic acid, capric acid, lauric acid, etc.)), a long chain fatty acid (carbon chain of 13-21 carbons (e.g., myristic acid, palmitic acid, stearic acid, arachidic acid, etc.)), a very long chain fatty acid (carbon chain of >21 carbons (e.g., behenic acid, lignoceric acid, cerotic acid, etc.)), and/or any suitable combinations thereof. In some embodiments, the acyl chain portion of the fatty acid comprises C₆-C₂₄ (e.g., C₆, C₈, C₁₀, C₁₂, C₁₄, C₁₆, C₁₈, C₂₀, C₂₂, C₂₄, or ranges therebetween). In some embodiments, a hydrophobic segment comprises multiple acyl chains or lipids. In some embodiments, the lipid is a saturated fatty acid selected from caprylic acid, capric acid, lauric acid, myristic acid, palmitic acid, stearic acid, arachidic acid, behenic acid, lignoceric acid, and cerotic acid. In some embodiments, the lipid is a saturated fatty acid selected from myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid.

In some embodiments, PAs comprise one or more linker moieties of other structural/functional moieties between the stapled peptide described herein and the hydrophobic segment. A variety of linker groups are contemplated, and suitable linkers could comprise, but are not limited to, alkyl groups, methylene carbon chains, ether, polyether, alkyl amide linker, a peptide linker, a modified peptide linker, a Poly(ethylene glycol) (PEG) linker, PABC (pamidobenzylocycarbonyl), functionalized PEG, polysaccharides, glycosaminoglycans, dendritic polymers (WO93/06868 and Tomalia et al. in Angew. Chem. Int. Ed. Engl. 29:138-175 (1990)), PEG-chelant polymers (W94/08629, WO94/09056 and WO96/26754), oligonucleotide linker, phospholipid derivatives, alkenyl chains, alkynyl chains, disulfide, or a combination thereof. In some embodiments, the linker is cleavable (e.g., enzymatically (e.g., cathepsin cleavable (e.g., Valine-Citruline), TEV protease cleavable, etc.), chemically, photoinduced, etc.

In other embodiments, the hydrocarbon stapled peptide may be packaged as a mucoadhesive polymer. The term “mucoadhesion,” as used herein, refers to the interaction between a mucin surface and a synthetic or natural polymer. Mucoadhesion differs from “bioadhesion” in that, in bioadhesion, a polymer is attached to a biological membrane instead of a mucus membrane. Drug delivery systems employing mucoadhesive polymers have recently been developed, in which a mucoadhesive polymer attaches to related tissue or to the surface coating of the tissue for the targeting of various absorptive mucosa such as ocular, nasal, pulmonary, buccal, vaginal, etc. Advantages of mucoadhesion drug delivery systems include, but are not limited to, the dosage form residing at the site of absorption for prolonged time, rapid absorption, increased drug bioavailability, and protection from drug degradation in the acidic environment of the gastrointestinal tract (Kavitha et al., Journal of Applied Pharmaceutical Science 01 (08): 37-42 (2011). Examples of suitable mucoadhesive polymers that may be used in the context of the inventive method include, but are not limited to, synthetic mucoadhesive polymers (e.g., cellulose derivatives, poly (acrylic acid) polymers, poly (hydroxyethyl methylacrylate), poly (ethylene oxide), poly (vinyl pyrrolidone), poly (vinyl alcohol)) and natural mucoadhesive polymers (e.g., ragacanth, sodium alginate, karaya gum, guar gum, xanthan gum, soluble starch, gelatin, pectin, chitosan, etc.). Other exemplary mucoadhesive polymers include poloxamer, hydroxypropyl methyl cellulose, methyl cellulose, hyaluronic acid, hydroxy propyl cellulose, gellan gum, carrageenan, hydrogels, lectins, thiolated polymers, bioadhesive nanopolymers, alginate-polyethylene glycol acrylate, and pluronics.

Other suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. In some embodiments, the carrier is a buffered saline solution. In other embodiments, the hydrocarbon stapled peptide may be formulated in a composition to protect the stapled peptide from damage prior to administration. The composition can be formulated to decrease the light sensitivity and/or temperature sensitivity of the hydrocarbon stapled peptide. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, or combinations thereof.

One or more cells expressing hACE2 may be contacted with the hydrocarbon stapled peptide, or a composition comprising same, in vitro or in vivo. The term “in vivo” refers to a method that is conducted within living organisms in their normal, intact state, while an “in vitro” method is conducted using components of an organism that have been isolated from its usual biological context. When the cells are contacted with the composition in vitro, the cell may be any suitable eukaryotic cell, such as cells of the vascular endothelia, renal and cardiovascular tissue, and epithelia of the small intestine and testes. When the cells are contacted with the composition in vivo, the composition may be administered to an animal, such as a mammal, particularly a human. In some embodiments, the human may be infected with a coronavirus (e.g., SARS-CoV-2) but is asymptomatic. Alternatively, the human may be infected with a coronavirus (e.g., SARS-CoV-2) and is exhibiting one or more symptoms of coronavirus infection (e.g., SARS-CoV-2). In other embodiments, the human may be at risk for infection or suspected of infection by a coronavirus (SARS-CoV-2).

Thus, the disclosure also provides a method of treating a coronavirus infection in a subject, which comprises administering to the subject a hydrocarbon stapled peptide, whereby the hydrocarbon stapled peptide binds to the receptor binding domain (RBD) of a coronavirus spike protein (S) and inhibits binding of the coronavirus to hACE2 expressed by cells in the subject, thereby treating the coronavirus infection in the subject.

The hydrocarbon stapled peptide, or a composition comprising same, may be administered to a human using any suitable administration technique and route. Suitable administration routes include, but are not limited to, oral, intravenous, intramuscular intraperitoneal, intranasal (e.g., via nebulizer), subcutaneous, pulmonary, transdermal, buccal, sublingual, or suppository administration. The hydrocarbon stapled peptide or composition comprising same ideally is suitable for parenteral administration. The term “parenteral,” as used herein, includes intravenous, intramuscular, subcutaneous, rectal, vaginal, and intraperitoneal administration.

Any suitable dose or amount of the hydrocarbon stapled peptide, or composition comprising same, may be administered to a mammal (e.g., a human), so long as the dose or amount is sufficient to inhibit binding of a coronavirus to hACE2 expressed by cells in the subject. To this end, the inventive method comprises administering an “effective amount” of the hydrocarbon stapled peptide. An “effective amount” refers to a sufficient amount, at dosages and for periods of time necessary, to achieve a desired biological result (e.g., blocking coronavirus infection). The effective amount may vary according to factors such as the age, sex, and weight of the individual. Ideally, an effective amount is an amount effective to prevent a coronavirus infection in the subject.

In some embodiments, the method comprises a single administration of the hydrocarbon stapled peptide, or composition comprising same, to the subject (e.g., a human). In other embodiments, the method comprises multiple administrations of the hydrocarbon stapled peptide, or composition comprising same, to the subject. Each of the multiple administrations may be separated by any suitable timeframe (e.g., at least about 1 week, 2 weeks, 4 weeks, 8 weeks, 12 weeks, 16 weeks, or more following the previous administration) to achieve maximum therapeutic benefit.

The following examples further illustrates the invention but, of course, should not be construed as in any way limiting its scope.

### EXAMPLE 1

This example describes the synthesis of hydrocarbon stapled peptides that bind to the receptor binding domain (RBD) of SARS-CoV-2 spike protein (S).

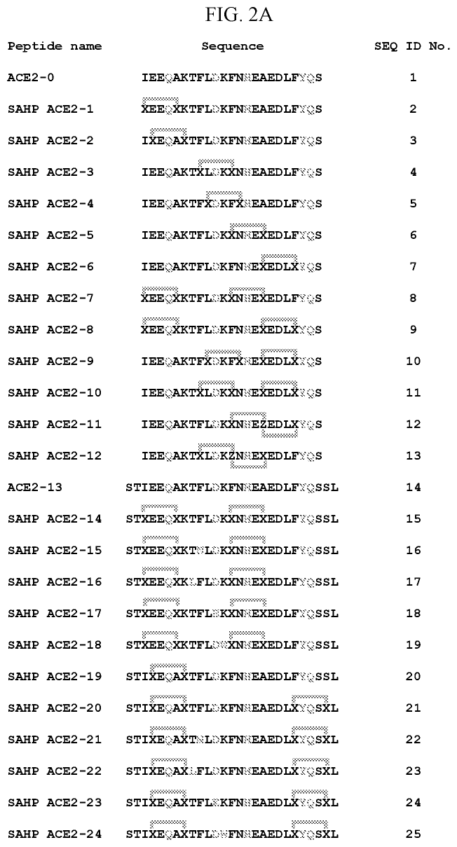

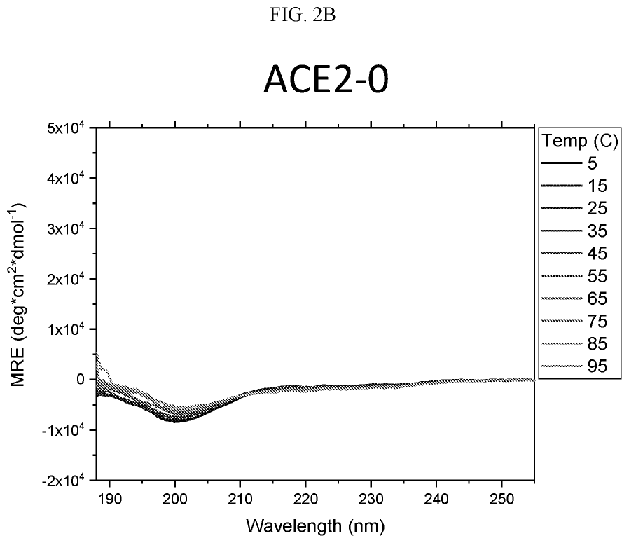

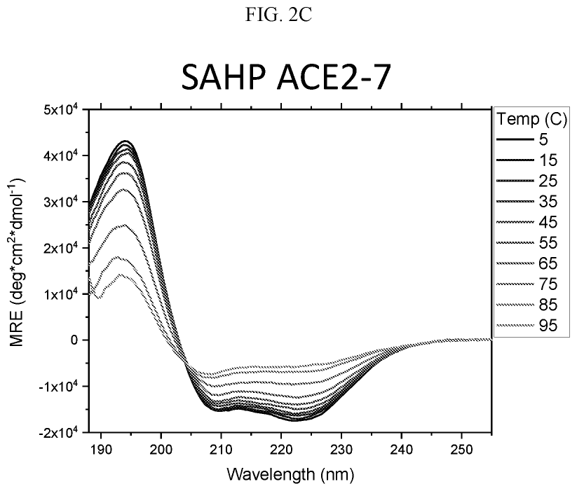

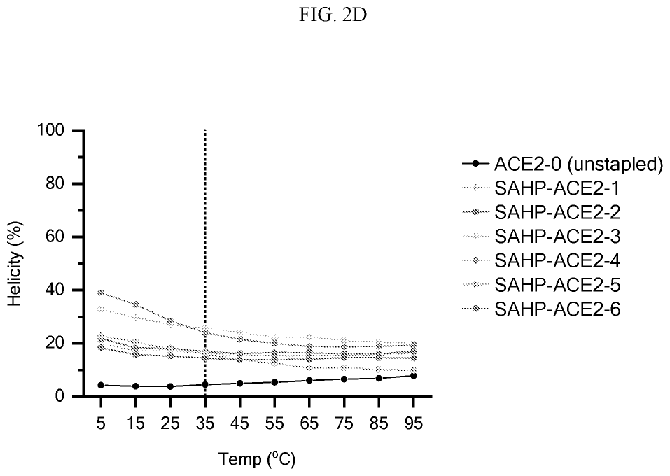

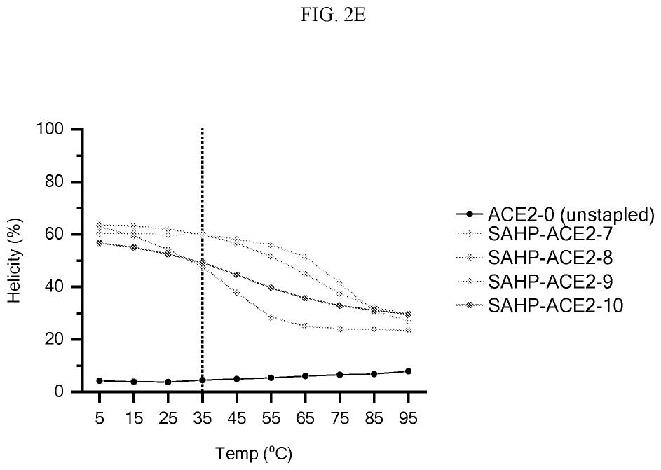

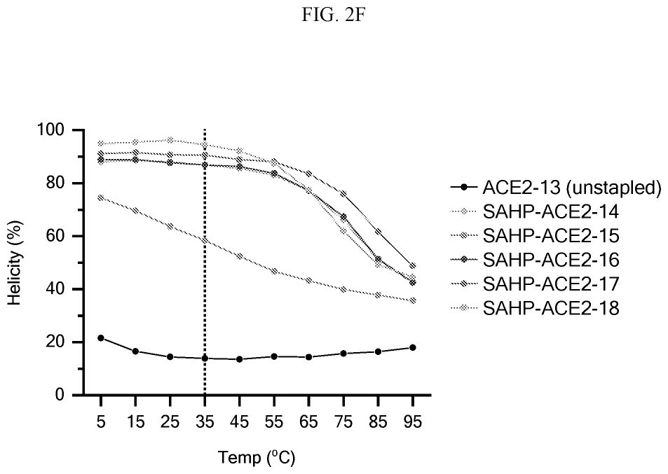

Peptides were synthesized, derivatized, purified, and reconstituted as previously described and using a standard Fmoc-based approach (Schnorenberg et al., ACS Omega, 3(10): 14144-14150 (2018); LaBelle et al., J Clin Invest., 122(6): 2018-2031 (2012); Hadji et al., Oncotarget, 10(58): 6219-6233 (2019); and Coin et al., Nat Protoc., 2(12): 3247-3256 (2007)). Briefly, peptides were prepared at a 0.2 mmol scale on Rink amide resin using an automated synthesizer (Prelude X, Protein technologies). The resins were swelled in dimethylformamide (DMF) for 30 minutes, then were double deprotected for 10 minutes with 20% piperidine in N-methyl-2-pyrrolidone (NMP). The coupling reaction was conducted for 40 minutes with 10 eq. of the appropriate amino acid dissolved in NMP at 0.3 M, 9.5 eq. HATU dissolved in NMP at 0.285 M, and 20 eq. diisopropylethylamine (DIPEA) dissolved in NMP at 0.6M. Five washes were performed in between steps with DMF. Hydrocarbon stapling was achieved by replacing the corresponding residues with unnatural amino acids ((S)-N-Fmoc-2-(4′-pentenyl) alanine and Fmoc-2,2-bis(4-pentenyl) glycine), followed by four times ring closing metathesis reaction catalyzed by 8 mL of 1st generation Grubbs catalyst (4 mg/mL in 1,2-dichloroethane) for 3 hours. After the last deprotection step, peptide acetylation was achieved by incubation with a solution of (by volume) 80% NMP and 20% acetic anhydride with additional 2% DIPEA for 20 minutes. Peptide cleavage was achieved by shaking peptide solutions for 2 hours in a cleavage cocktail comprising (by volume) 95% trifluoroacetic acid (TFA), 2.5% triisoproylsilane (TIIS), and 2.5% Milli-Q water. The final peptide was precipitated by adding the cleavage cocktail and cleaved peptide to ice cold diethyl ether. The mixture was centrifuged, and the supernatant discarded. The resulting peptide was then dissolved in a 1:1 mixture of acetonitrile (ACN) and Milli-Q water for HPLC purification and lyophilization. The stapled peptide sequences are shown in FIG. 2A. Amino acids, DIPEA, Grubbs catalyst, and resin were purchased from Millipore Sigma. HATU and other solvents were purchased from Protein Technologies. All the chemicals were used as received. All peptides were purified by liquid chromatography/mass spectrometry (LC/MS) to more than 95% purity and quantified by amino acid analysis.

### EXAMPLE 2

This example describes an analysis of the secondary structure of the hydrocarbon stapled peptides.

Secondary structure of the peptides prepared in Example 1 was analyzed using a CD spectropolarimeter (J-815, JASCO Corporation). Sample solutions were prepared at 25 μM concentration in 10 mM phosphate buffers and transferred into an absorption cuvette with 1 mm path length (110-QS, Hellma, Inc.). Pure buffer solutions were used for the background correction. Unless otherwise specified, for full wavelength scans, sample spectra were recorded from 185-255 nm at desired temperatures, with a scanning rate of 50 nm/minute, and averaged over three wavelength scans. Data points for the wavelength-dependent CD spectra were recorded at every 0.05 nm with a 1 nm bandwidth and a 4 second response time for each data point. To measure any potential denaturation and hysteresis, full wavelength scans were performed while both increasing and decreasing temperature between 5° C. and 95° C. at 10° C. intervals, the heating/cooling rate was set to 4° C./min; and 1 minute was allowed after reaching each temperature point for sample equilibration. The CD data were converted to mean residue ellipticity, [θ] (deg cm² dmol⁻¹) using the formula:

$$[ θ ] ( deg \cdot {cm}^{2} \cdot {dmol}^{- 1} ) = \frac{θ ( millidegree )}{L ( mm ) \times c ( M ) \times N}$$

in which θ is the measured ellipticity in millidegree, L is the pathway length of CD cuvette in millimeter, c is the peptide solution molar concentration in mol/L, and N is the number of amino acid residues. The values of θ at 222 nm were used to monitor temperature-dependent behavior and converted to a percent alpha helicity using the formula:

$$\% Helicity = \frac{100 \times {[ θ ]}_{222}}{{[ θ ]}_{222}^{max}} where$$

$${[ θ ]}_{222}^{max} = - 40 , 000 \times [ 1 - ( \frac{2.5}{number of amino acid residues} ) ] + 100 \times T$$

with T measured in ° C. Table 1 shows that hydrocarbon staple position and quantity (SAHP ACE 2-7 is double-stapled) greatly influences the stability of the α-helical secondary structure.

TABLE 1

| Peptide | % helicity at 35° C. |
|---|---|
| ACE2-0 | 4.05 |
| SAHP ACE2-1 | 7.98 |
| SAHP ACE2-2 | 10.55 |
| SAHP ACE2-3 | 23.06 |
| SAHP ACE2-4 | 21.60 |
| SAHP ACE2-5 | 14.44 |
| SAHP ACE2-6 | 13.00 |
| SAHP ACE2-7 | 44.70 |

Additional results of this analysis are shown in FIGS. 2B-2D.

### EXAMPLE 3

This example demonstrates the proteolytic stability of the hydrocarbon stapled peptides described herein.

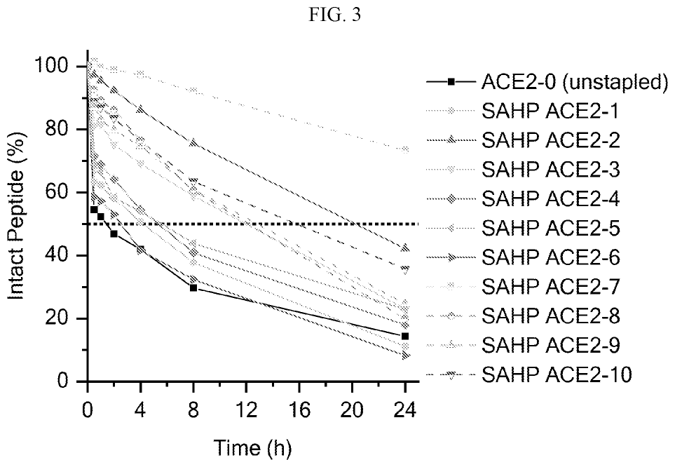

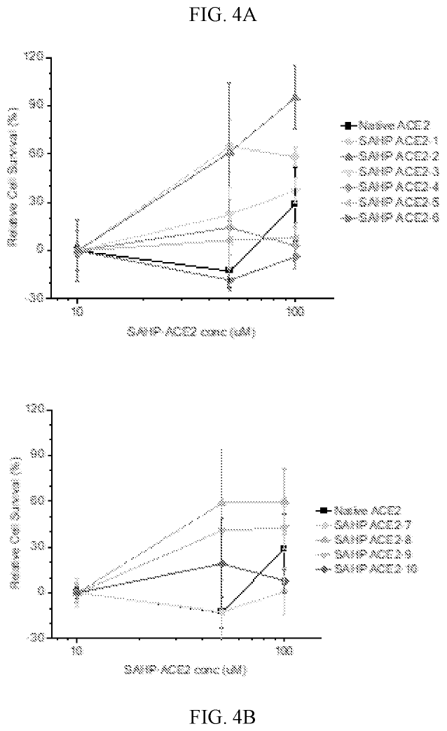

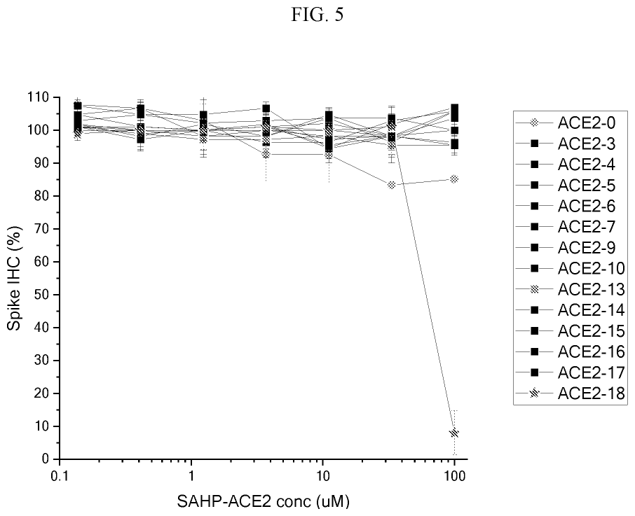

Pure peptides were prepared in dimethyl sulfoxide DMSO to a concentration of 20 mM as stock solutions and diluted to the desired working concentration of 0.2 mM using PBS. A slurry form of immobilized trypsin (ThermoFisher Scientific) was used to assess proteolytic stability. Trypsin slurry was first equilibrated to room temperature, vortexed, and washed twice with 10 eq volume of PBS and by centrifuging at 17 g for 10 minutes. A working solution of trypsin slurry was made by diluting the washed stock slurry to 1/10 of its concentration with PBS. 2004 of peptide solution and 1004 of trypsin solution were mixed in 7 vials in parallel to be sampled after incubation at 37° C. for 0 minutes, 30 minutes, 1 hour, 2 hours, 4 hours, 8 hours, and 24 hours. Samples were quenched at each desired time point by adding 504 of acetonitrile (ACN) to the solution before centrifugation at 17 g for 10 minutes. The supernatant was then sampled and subjected to LC/MS analysis (Agilent Technologies). The separation was achieved using an analytical C18 column (Waters Corporation) with linear elution gradient from 80%/20% of water (0.1%TFA)/ACN to 100% ACN and monitored with UV absorbance at 280 nm. The amount of intact peptide was analyzed through UV peak integration. The molecular weight of elute was analyzed using electrospray ionization mass spectrometry (ESI-MS). The results of this analysis are shown in FIG. 3.

### Sequences

TABLE 2
Generation I Sequences

| | |
|---|---|
| ACE2-0 (SEQ ID NO: 1): | IEEQAKTFLDKFNHEAEDL |
| | FYQS |
| SAHP ACE2-1 (SEQ ID NO: 2): | XEEQXKTFLDKFNHEAEDL |
| | FYQS |
| SAHP ACE2-2 (SEQ ID NO: 3): | IXEQAXTFLDKFNHEAEDL |
| | FYQS |
| SAHP ACE2-3 (SEQ ID NO: 4): | IEEQAKTXLDKXNHEAEDL |
| | FYQS |
| SAHP ACE2-4 (SEQ ID NO: 5): | IEEQAKTFXDKFXHEAEDL |
| | FYQS |
| SAHP ACE2-5 (SEQ ID NO: 6): | IEEQAKTFLDKXNHEXEDL |
| | FYQS |
| SAHP ACE2-6 (SEQ ID NO: 7): | IEEQAKTFLDKFNHEXEDL |
| | XYQS |
| SAHP ACE2-7 (SEQ ID NO: 8): | XEEQXKTFLDKXNHEXEDL |
| | FYQS |
| SAHP ACE2-8 (SEQ ID NO: 9): | XEEQXKTFLDKFNHEXEDL |
| | XYQS |
| SAHP ACE2-9 (SEQ ID NO: 10): | IEEQAKTFXDKFXHEXEDL |
| | XYQS |
| SAHP ACE2-10 (SEQ ID NO: 11): | IEEQAKTXLDKXNHEXEDL |
| | XYQS |
| SAHP ACE2-11 (SEQ ID NO: 12): | IEEQAKTFLDKXNHEZEDL |
| | XYQS |
| SAHP ACE2-12 (SEQ ID NO: 13): | IEEQAKTXLDKZNHEXEDL |
| | FYQS |

TABLE 3
Generation II/III Sequences. ACE2-13 is an
unstapled control with 2 extra amino acids on
each end. Different point mutations are included
in generation II/III peptides based on in silico
data. Two different double stapling strategies
are compared to explore the significance of
chain flexibility in the middle
(See FIG. 2A for stapling strategies).

| | |
|---|---|
| SAHP ACE2-13 (SEQ ID NO: 14): | STIEEQAKTFLDKFNHEA |
| | EDLFYQSSL |
| SAHP ACE2-14 (SEQ ID NO: 15): | STXEEQXKTFLDKXNHEX |
| | EDLFYQSSL |
| SAHP ACE2-15 (SEQ ID NO: 16): | STXEEQXKTNLDKXNHEX |
| | EDLFYQSSL |
| SAHP ACE2-16 (SEQ ID NO: 17): | STXEEQXKLFLDKXNHEX |
| | EDLFYQSSL |
| SAHP ACE2-17 (SEQ ID NO: 18): | STXEEQXKTFLEKXNHEX |
| | EDLFYQSSL |
| SAHP ACE2-18 (SEQ ID NO: 19): | STXEEQXKTFLDWXNHEX |
| | EDLFYQSSL |
| SAHP ACE2-19 (SEQ ID NO: 20): | STIXEQAXTFLDKFNHEA |
| | EDLFYQSSL |
| SAHP ACE2-20 (SEQ ID NO: 21): | STIXEQAXTFLDKFNHEA |
| | EDLXYQSXL |
| SAHP ACE2-21 (SEQ ID NO: 22): | STIXEQAXTNLDKFNHEA |
| | EDLXYQSXL |
| SAHP ACE2-22 (SEQ ID NO: 23): | STIXEQAXLFLDKFNHEA |
| | EDLXYQSXL |
| SAHP ACE2-23 (SEQ ID NO: 24): | STIXEQAXTFLEKFNHEA |
| | EDLXYQSXL |
| SAHP ACE2-24 (SEQ ID NO: 25): | STIXEQAXTFLDWFNHEA |
| | EDLXYQSXL |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inhibiting binding of a coronavirus to a human angiotensin-converting enzyme 2 (hACE2) expressed on the surface of a cell, which comprises contacting one or more cells expressing hACE2 with a hydrocarbon stapled peptide, whereby the hydrocarbon stapled peptide binds to the receptor binding domain (RBD) of a coronavirus spike protein (S) and inhibits binding of the coronavirus to hACE2.

2. The method of claim 1, wherein the coronavirus is coronavirus OC43, coronavirus 229E, coronavirus NL63, coronavirus HKU1, MERS-CoV, SARS-CoV, or SARS-CoV-2.

3. The method of claim 1 or claim 2, wherein the coronavirus is SARS-CoV-2.

4. The method of any one of claims 1-3, wherein the hydrocarbon stapled peptide is a hACE2 peptidomimetic.

5. The method of any one of claims 1-4, wherein the hydrocarbon stapled peptide mimics the alpha-helical domain of hACE2.

6. The method of any one of claims 1-5, wherein the hydrocarbon stapled peptide is single- or double-stapled.

7. The method of any one of claims 1-6, wherein the hydrocarbon stapled peptide comprises at least 70% sequence identity with an amino acid sequence of SEQ ID NO: 1, SEQ ID NO: 2, SEQ ID NO: 3, SEQ ID NO: 4, SEQ ID NO: 5, SEQ ID NO: 6, SEQ ID NO:
7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO:
23, SEQ ID NO: 24, or SEQ ID NO: 25.

8. The method of claim 7, wherein the hydrocarbon stapled peptide comprises an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

9. The method of any one of claims 1-8, wherein the hydrocarbon stapled peptide is encapsulated in a polymersome.

10. The method of any one of claims 1-8, wherein the hydrocarbon stapled peptide is packaged as a peptide amphiphile or a mucoadhesive polymer.

11. The method of any one of claims 1-10, wherein the one or more cells expressing hACE2 are present in a human.

12. The method of claim 11, wherein contacting one or more cells expressing hACE2 with a hydrocarbon stapled peptide comprises administering the hydrocarbon stapled peptide to the human via intranasal, intravenous, or oral administration.

13. A method of treating a coronavirus infection in a subject, which comprises administering to the subject a hydrocarbon stapled peptide, whereby the hydrocarbon stapled peptide binds to the receptor binding domain (RBD) of a coronavirus spike protein (S) and inhibits binding of the coronavirus to hACE2 expressed by cells in the subject, thereby treating the coronavirus infection in the subject.

14. The method of claim 13, wherein the coronavirus is coronavirus OC43, coronavirus 229E, coronavirus NL63, coronavirus HKU1, MERS-CoV, SARS-CoV, or SARS-CoV-2.

15. The method of claim 13 or claim 14, wherein the coronavirus is SARS-CoV-2.

16. The method of any one of claims 13-15, wherein the hydrocarbon stapled peptide is a hACE2 peptidomimetic.

17. The method of any one of claims 13-16 wherein the hydrocarbon stapled peptide mimics the alpha-helical domain of hACE2.

18. The method of any one of claims 13-1617 wherein the hydrocarbon stapled peptide is single- or double-stapled.

19. The method of any one of claims 13-18, wherein the hydrocarbon stapled peptide comprises at least 70% sequence identity with an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

20. The method of claim 19, wherein the hydrocarbon stapled peptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

21. The method of any one of claims 13-20, wherein the hydrocarbon stapled peptide is encapsulated in a polymersome.

22. The method of any one of claims 13-20, wherein the hydrocarbon stapled peptide is packaged as a peptide amphiphile or a mucoadhesive polymer.

23. The method of any one of claims 13-22, wherein the hydrocarbon stapled peptide is delivered to the human via intranasal, intravenous, or oral administration.

24. Use of a hydrocarbon stapled peptide for the treatment of a coronavirus infection in a subject.

25. A composition comprising a hydrocarbon stapled peptide and a pharmaceutically acceptable carrier, wherein the hydrocarbon stapled peptide comprises at least 70% sequence identity with an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

26. A composition comprising a hydrocarbon stapled peptide and a pharmaceutically acceptable carrier, wherein the hydrocarbon stapled peptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

27. The composition of claim 25 or 26, which is in the form of a polymersome, a peptide amphiphile, or a mucoadhesive polymer.
